(19) United States
(12) Patent Application Publication
(10) Pub. No.: US 2009/0185992 A1
(43) Pub. Date: Jul. 23, 2009

(54) PROCESS FOR PRODUCING IRON OXIDE COATED PEARLESCENT PIGMENTS

(76) Inventors: Christelle Conan, Cincinnati, OH (US); Aaron M. Hollman, Union, KY (US); Philippe Schottland, West Chester, OH (US); Stephane Nicolas, Fleurines (FR); Marguerite Debacker, Court Saint Etienne (BE); Aurelie Antonowicz, Gembloux (BE); Kenneth A. Fields, II, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 12/016,341

(22) Filed: Jan. 18, 2008

(51) Int. Cl. C09C 1/00; C09D 11/00; C09D 1/00; A61K 8/19; A61Q 1/02; A61Q 1/06; A61Q 3/02; A61Q 5/06; C11D 3/14

(52) U.S. Cl. 424/61; 106/456; 106/418; 106/415; 106/425; 106/439; 106/450; 106/453; 106/31.9; 106/286.3; 424/646; 424/63; 424/64; 424/70.1; 510/130

(57) ABSTRACT

A pearlescent pigment, wherein the pigment is an inorganic material and the color of a homogeneous coating of the pigment, measured over a black background has a CIELAB hue angle, h_{ab}, from about 40 to about 320 degrees, wherein L* is from about 35 to about 80, and the chroma value is less than 5. A substrate is coated with iron oxide by oxidizing an iron salt to form a pigment. The pigment may be made by a process of coating iron oxide on a substrate by oxidizing an iron salt. During the coating of the iron oxide, the pH of the mixture is increased. The coating formed has a Fe(III)/Fe(II) ratio of greater than 2. The pigments may be used in a variety of applications including coating, ink, plastic, and cosmetic compositions.

### BACKGROUND OF THE INVENTION

The present invention relates, in general, to multi-colored lustrous pearlescent pigments.

Along with gem stones (e.g., diamond, ruby, emerald, topaz, opal, jade), and precious metals (e.g., gold, silver, platinum), pearls are among the most prized possessions (or luxury items) for human beings for millenniums. Beside their natural beauty, the brilliant color and luster, they are often associated with social status and level of well-being. As a result, and not surprisingly, the trend of cosmetics makeup is to emulate or recreate these “natural” and “aesthetic” appearances of pearl, gem and precious metals with less expensive materials such as interference pigments (e.g., metal oxide coated mica). The most common types of pearlescent pigments are micronized titanium dioxide, metal oxide coated mica, metal oxide coated alumina, metal oxide coated silica, basic lead carbonate, bismuth oxychloride, and natural fish silver.

Metal oxide coated mica pigments are characterized by excellent optical, chemical, mechanical, toxicological, and environmental properties. Natural or synthetic mica, and alternative supports, such as aluminum flakes, or SiO₂ platelets, can be used alone, or as a support for titanium dioxide, iron oxide (Fe₂O₃ or Fe₃O₄), iron ferrocyanide (Iron Blue or Prussian Blue), tin oxide, and chromium oxide. The color space defined by these coated mica-based pigments is based on the type of coating (e.g. metal oxide, colorant, etc.) used, the layer thickness, and the number of coated layers.

Metal oxide coated mica pigments may be made by coating a mica substrate with an iron(II) containing layer by simultaneously adding iron(II) salt solution and a separate solution of an oxidizing agent to a substrate as described in U.S. Pat. No. 4,867,793. Another procedure for making a metal oxide coated mica pigment is by coating a layer of iron(II) oxide on a substrate followed by a reduction as described in U.S. Pat. No. 3,926,659.

Among the natural pearls, the most expensive are black pearls, which come with various undertone and color flops. To faithfully emulate this aesthetic optical effect in cosmetic makeup is one of the top challenges facing a cosmetic pigment maker and formulator. The traditional approach to these pigments is to blend dark solid-color inorganic pigment (e.g., carbon black) with white platy pearlescent pigments (e.g., TiO₂ coated mica, TiO₂ coated borosilicate, TiO₂ coated alumina). The platy interference pigment provides the luster, brilliance (reflection), transparency and depth of field. The solid-color pigment(s) provide(s) the dark undertone and surface coverage. However, this type of blend usually appears to be much “dirtier”, “lack luster”, and “lack transparency” compared to the natural pearl. The primary reason for that is fouling of the smooth surface of white pearlescent pigment by the solid-color pigment granules, which leads to light scattering and disruption of light interference.

Metal oxide coated platelet pigments may be magnetic or exhibit magnetic susceptibility. When placed into a liquid coating, regions of the coated pigment may be aligned by an externally applied magnetic field and produce a goniochromatic, or angle dependent optical effect. This effect may be used to create an impression of a two- or three-dimensional image. After the pigments have been aligned, the coating may be cured to solidify the optical effect. Examples of pigments and methods of aligning them are discussed in U.S. Pat. No. 6,589,331, U.S. Pat. No. 6,902,807, U.S. Pat. No. 5,223,360, U.S. Pat. No. 6,759,097, and U.S. Pat. No. 7,258,900. However, the magnetic pigments are significantly limited in terms of color space. The typical colors available are metallic black, grey shades, or bichromic shades characterized by a black or reddish brown absorbance color combined with a weak interference color.

A need exists to expand the existing color space of metal oxide coated pigments to more vibrant, lustrous, bright colored shades, using a processing method that allows for optimal control of color and opacity. In addition, a need exists for more colorful, bright magnetic pigments that have a larger color contrast between aligned and non-aligned pigments.

### BRIEF SUMMARY OF THE INVENTION

The invention overcomes the above-noted and other deficiencies of the prior art by a pearlescent pigment made by the process comprising providing a substrate, coating iron oxide on the substrate by oxidizing an iron salt with an oxidant in a mixture while increasing the pH of the mixture from acidic to basic.

Another aspect of the invention is a process for making a pearlescent pigment comprising providing a substrate, coating iron oxide on the substrate by oxidizing an iron salt with an oxidant in a mixture while increasing the pH from acid to basic.

Another aspect of the invention is a pearlescent pigment, wherein the pigment is an inorganic material and the color of a homogeneous coating of the pigment, measured over a black background, has a CIELAB hue angle, h_{ab}, from about 40 to about 320 degrees, wherein L* is from about 35 to about 80, and the chroma value is less than 5.

Another aspect of the invention is a cosmetic composition comprising a pearlescent pigment made by the process comprising providing a substrate, coating iron oxide on the substrate by oxidizing an iron salt with an oxidant in a mixture while increasing the pH of the mixture from acidic to basic.

Another aspect of the invention is a coating or ink composition containing a pearlescent pigment made by the process comprising providing a substrate, coating iron oxide on the substrate by oxidizing an iron salt with an oxidant in a mixture while increasing the pH of the mixture from acidic to basic.

These and other objects and advantages of the present invention shall be made apparent from the accompanying description.

### DETAILED DESCRIPTION OF THE INVENTION

The present invention relates to a pearlescent pigment made by the process comprising providing a substrate, coating iron oxide on the substrate by oxidizing an iron salt with an oxidant in a mixture while increasing the pH of the mixture from acidic to basic.

The iron(II) in solution will form iron oxide that coats the substrate, by a reaction of oxidation followed by hydrolysis. At a low pH, the oxidation reaction of Fe(II) produces FeOOH which coats the substrate. When the pH is around 8 or higher, magnetite, Fe₃O₄ is produced and coats the substrate. Controlling the pH during the process allows control of the ratio of Fe(III)/Fe(II) in the iron oxide of the coating. Coatings with non-conventional Fe(III)/Fe(II) ratios can be obtained because the coating can be a mixture of FeOOH and Fe₃O₄.

Examples of oxidants that may be used to oxidize the iron(II) are nitrates and chlorates. One example is potassium nitrate. The oxidizing agent may be added to the coating mixture as a powder or in a solution. The oxidant may be added all at once, continuously, or batch-wise during the coating.

The coating process starts with a low pH (acidic), which is increased to a high pH (basic) during the process of forming the iron oxide layer. The pH may start in the range of about 0.2 to about 4, and increase to a range of about 7 to about 12. Examples of a base that may be used to increase the pH are NaOH, KOH, LiOH, urea, and ammonia.

Examples of iron salts are ammonium iron(II) sulfate, iron(II) halides, and iron(II) sulfate, iron(II) acetates, iron(II) carbonates, iron(II) phosphates. In one embodiment the iron salts are selected from the group consisting of iron(II) halides and iron(II) sulfates.

One embodiment is a process for making a pearlescent pigment comprising providing a substrate, coating iron oxide on the substrate by oxidizing an iron salt with an oxidant in a mixture while increasing the pH from acidic to basic.

In one embodiment, the pearlescent pigment comprises a substrate and a first layer or coating, wherein the first layer comprises iron oxide, wherein the iron(III)/iron(II) ratio of the iron oxide is greater than about 2, from about 2 to about 6, from about 2.5 to about 6, or about 4. The iron oxide layer may be one layer, or may comprise multiple layers of iron oxide. The iron of the iron oxide for one layer, may be less than about 30% of the weight of the pigment. By coating several successive layers, the total amount of iron of the iron oxide may be up to about 50% of the weight of the pigment. In one embodiment the iron of the iron oxide for one layer may be less than about 15% of the weight of the pigment. In another embodiment, the pigment comprises several successive layers of iron oxide, wherein the total amount of iron of the iron oxide may be up to about 50% of the weight of the pigment. In another embodiment there is only one layer of iron oxide wherein the iron of the iron oxide is less than about 12% of the weight of the pigment.

The interference color of the substrate determines the final pigment color. Direct precipitation of iron(II) oxide and magnetite produces a layer on the substrate, but the thickness of the layer doesn't induce “travel” of the color, or if it does, only a small “travel” of the color. As example, the color won't pass from red to blue on the same substrate. The color of the interference pigment may be accentuated and darkened by increasing the thickness of the iron oxide layer. But the strength of the shade may be finely tuned by controlling parameters like the temperature and the pH of the coating mixture.

Iron oxide coated substrates exhibit intensely colored pearlescent pigments with high luster. Varying the substrate, the iron oxide layer thickness, and the amount of Fe(II) and Fe(III) in the layer will change the color, luminosity, and transparency of the pigment. The mean thickness of the first layer may be greater than about 10 nm, from about 1 nm to about 350 nm, from about 10 nm to about 350 nm, or from about 10 nm to about 250 nm.

In one embodiment, the pigment may comprise a second layer located between the substrate and the first layer, wherein the second layer has a refractive index of greater than about 1.6 or less than about 1.4. The second layer may have a refractive index equal to or greater than about 1.8. Examples of compounds that may be used as the second layer are: TiO₂, Fe₂O₃, FeOOH, ZrO₂, SnO₂, Cr₂O₃, SiO₂, BiOCl, and ZnO. The second layer may comprise one or more materials. The second layer may be TiO₂. The second layer may be an iron oxide, such as Fe₂O₃, Fe₃O₄, FeOOH, FeO, and Fe(OH)₃. The methods of deposition (or precipitation) of FeOOH or Fe(OH)₃ onto substrates are well known in the literature, for example as shown in Dyes and Pigments, 58 (2003), 239-244, U.S. Pat. No. 3,926,659, and in many scientific papers and patents particularly by Merck, Engelhard, and BASF. The mean thickness of the second layer may be from about 10 nm to about 800 nm, or from about 10 nm to about 350 nm.

In one embodiment, the pigment may comprise a third layer located between the substrate and second layer, wherein the third layer has a refractive index of greater than about 1.6 or less than about 1.4. The second layer may have a refractive index equal to or greater than about 1.8. Examples of compounds that may be used as the third layer are: TiO₂, Fe₂O₃, FeOOH, ZrO₂, SnO₂, Cr₂O₃, SiO₂, BiOCl, and ZnO. The third layer may comprise one or more materials. The third layer may be TiO₂. The third layer may be an iron oxide, such as Fe₂O₃, Fe₃O₄, FeOOH, FeO, and Fe(OH)₃. The mean thickness of the third layer may be from about 50 nm to about 800 nm, or from about 100 nm to about 600 nm.

In another embodiment, titanium oxide coated mica pigments exhibit pearlescent effects resulting from reflection and light interference. The interference color and luster is dependent on the thickness of the TiO₂ surface layer and its corresponding surface roughness. This initial interference color of the pigment, prior to the coating of the first layer is apparent when viewed against a black background.

In order to improve the light, water repellency, weather stability, texture, and dispersion ability, it is frequently advisable to subject the finished pigment to surface treatment, depending on the area of application. Examples of surface treatments are methicone(poly(oxy(methylsilylene))), metal soap, fatty acid, hydrogenated lecithin, dimethicone(polydimethylsiloxane), fluorinated compounds, amino acids, N-acylamino acids, glyceryl rosinates, silanes, and combinations. Many of the processes are described in U.S. Pat. No. 6,790,452; U.S. Pat. No. 5,368,639; U.S. Pat. No. 5,326,392; U.S. Pat. No. 5,486,631; U.S. Pat. No. 4,606,914; U.S. Pat. No. 4,622,074; German Patent 22 15 191; DE-A 31 51 354; DE-A 32 35 017; DE-A 33 34 598; DE 40 30 727 A1; EP 0 649 886 A2; WO 97/29059; WO 99/57204; U.S. Pat. No. 5,759,255; EP 0090259; EP 0 634 459; WO 99/57204; WO 96/32446; WO 99/57204; U.S. Pat. No. 5,759,255; U.S. Pat. No. 5,571,851; WO 01/92425; U.S. Pat. No. 5,472,491; J. J. Ponjee, Philips Technical Review, Vol. 44, No. 3, 81 ff; and P. H. Harding J. C. Berg, J. Adhesion Sci. Technol. Vol. 11 No. 4, pp. 471-493. This post-coating may further increase the chemical stability or simplify handling of the pigment, in particular incorporation into various media. In order to improve the wettability, dispersibility and/or compatibility with the user media, functional coatings of Al₂O₃ or ZrO₂ or mixtures thereof may be applied to the pigment surface.

In one embodiment, coupling agents may be used to form an outer layer on the pearlescent pigment. Suitable coupling agents are disclosed in EP 632 109. Examples include, silanes, zirconium aluminates, zirconates, and titanates. The silanes may possess the structure Y—(CH₂)ₙ—SiX₃ in which n is 2-8, Y is an organofunctional group, e.g. an amino, methacrylic, vinyl, alkyl, aryl, halogen and/or epoxy group, and X is a silicon-functional group which following its hydrolysis reacts with active sites of an inorganic substrate or by condensation with other silicon compounds. This group Y may comprise, for example a hydroxy, a halogen or an alkoxy group.

In addition to these substantially hydrophilic coupling agents, it is also possible to use hydrophilic silanes, especially the aryl-, alkyl- and fluoroalkyl-substituted di- and trimethoxysilanes. These include, for example, phenethyltrimethoxysilane, propyltrimethoxysilane, butyltrimethoxysilane, isobutyltrimethoxysilane, hexyltrimethoxysilane, octyltrimethoxysilane, 1H, 1H, 2H, 2H-perfluorodecyltrimethoxysilane and (3,3,3-trifluoropropyl)methyldimethoxysilane. The concentration of coupling agents may be 0.2-1.2% by weight with respect to the base pigment.

In one embodiment, the pigment is platelet-like and the mean particle size is from about 1 μm to about 750 μm. In another embodiment, the mean particle size may be from about 5 μm to about 300 μm, or from about 10 μm to about 100 μm. In one embodiment, the pigment has a mean thickness of about 0.05 to about 5 μm. In another embodiment, the pigment has a mean thickness of about 0.1 μm to about 5 μm, or from about 0.2 μm to about 2 μm.

The synthesis of a particular colored pearlescent pigment begins with selection of the proper substrate material. The substrate may be comprised of natural mica, synthetic mica, glass flakes, talc, kaolin, Al₂O₃ platelets, SiO₂ platelets, TiO₂ flakes, BiOCl, borosilicate, synthetic alumina, and boron nitride. Such substrates may be multilayer materials, i.e. include materials of different refractive indices. The substrate may comprise mica. The pearlescent pigment may comprise a mixture of different substrates. Furthermore, the substrate may be made of identical or different flakes which differ in particle size.

The basic principle of deposition is as follows: Fe(II) precursors such as iron(II) chloride, or iron(II) sulfate, are dissolved in an acidic medium containing the substrate and an oxidant. As the pH is increased by the addition of bases such KOH, NaOH, LiOH, urea, or ammonia; some Fe(II) is oxidized to Fe(III) and precipitated out as either colloidal FeOOH or Fe₃O₄ particles, or dense aggregates depending on the pH control profile, temperature and concentration, and in some case the presence of electrical field. In the presence of substrates with affinity to iron oxides, the colloidal particles can quickly form a uniform film on the substrate.

The traditional precipitation process utilizing KOH and NaOH is called a heterogeneous hydrolysis process. However, more recently, a newer process called homogenous hydrolysis, utilizes urea as an in situ generated base. This process is said to produce a smoother and more transparent metal oxide film as shown in Dyes and Pigments 58 (2003) 239-244, and Materials Research Bulletin (1999), vol. 34, no. 6, 905-914. The use of ferric sulfate may produce coarser particles, and weaker luster than ferric chloride. A computer-programmed rate-control heater may be used to better control the decomposition rate of urea, which in turn controls the rate of ammonia (base) generation.

In one embodiment the pearlescent pigment is an inorganic material, and the color of a homogeneous coating of the pigment, measured over a black background, has a CIELAB hue angle, h_{ab}, from about 40 to about 320 degrees, wherein L* is from about 35 to about 80, and the chroma value is less than 5.

In one embodiment the pearlescent pigment is blue, with a CIELAB hue angle, h_{ab}, from about 170 to about 275 degrees, measured over a black background using a D65 illuminant and a 10 degree observer. Blue pearlescent pigments may be produced that do not contain iron blue (ferric ferrocyanide), allowing the pigment to be used in cosmetic applications involving the lip area, such as lip gloss, lipstick, and other lip formulations. In addition, the blue pearlescent pigments are more stable than ferric ferrocyanide, which decomposes in alkaline pH resulting in pigment bleeding and marked changes in pigment color. Another advantage of the blue pearlescent pigments is that they do not restrict the pigment to the color space defined by ferric ferrocyanide. Iron Blue is a powerful colorant that only allows the pigment designer to formulate pigments within a well defined, narrow color space and restricts formulation flexibility, see Dyes and Pigments 56 (2003) 93-98.

In one embodiment the pearlescent pigment is green, with a CIELAB hue angle, h_{ab}, from about 80 to about 170 degrees, measured over a black background using a D65 illuminant and a 10 degree observer. Green pearlescent pigments may be produced that do not contain chromium oxide, allowing the pigment to be used in cosmetic applications involving the lip area, such as lip gloss, lipstick, and other lip formulations. Most commercially available green pearlescent pigments are based on chromium oxide deposition, U.S. Pat. No. 6,485,556.

The pigments may be magnetic or exhibit magnetic susceptibility. In one embodiment the magnetic susceptibility is greater than about 0.05×10⁻⁵ m³/kg. In another embodiment the magnetic susceptibility is greater than about 0.1×10⁻⁵ m³/kg. In another embodiment the magnetic susceptibility is greater than about 10⁻⁵ m³/kg. In fluid-based systems, such as liquid coatings or uncured plastic preparations containing these pigments, an applied magnetic field may be used to align pigments in specific regions of the coating to create images that appear to be three-dimensional. After the pigments have been aligned, the coating may be cured to solidify the image.

The three-dimensional effect is produced by the pigment particles aligned at non-parallel or intermediate angles with respect to the coating surface. In an applied electric field, the high aspect ratio platelets will align themselves such that the longest dimension of the platelet (namely, the platelet width) aligns itself along the magnetic field lines. The ability to reorient the colored particles allows them to be manipulated to specific angles resulting in a controlled three-dimensional appearance. In regions of where the field lines are perpendicular to the observer, the platelet particles will be perpendicular to the observer resulting in a jet black appearance. This extremely dark appearance is due to light scattering at the particle edges and the absence of a reflective surface. In regions devoid of an applied magnetic force, the particles align more substantially parallel to the applied coating surface resulting in the intensely colored appearance. Appling a magnetic field parallel to the coating surface will orient more of the pigments parallel to the surface resulting in a even more intensely colored appearance. In one embodiment the pearlescent pigment has a ΔE* of magnetically aligned and non-aligned homogeneous coatings of the pigment, measured over a white background, not less than about 10.

In one embodiment a cosmetic composition contains the pearlescent pigment. The cosmetic composition may be useful for make-up products for the skin, the eyes, or hair. Examples of compositions intended as make-up for the skin include eye shadows, eye liners, mascaras, body or face powder, foundations, blushes, colored creams, nail polish, lipsticks, lip gloss, hair or body gel, hair or body wash, cover sticks, lotion, concealer and foundation. Examples of cosmetic applications involving the lip area, are lip gloss, lipstick, and other lip compositions. Nail polish may be referred to as nail varnish, or nail enamel.

Pearlescent pigments may be used to produce a makeup cosmetic composition as described in U.S. Pat. No. 6,663,852, U.S. Pat. No. 6,451,294, and U.S. Pat. No. 6,280,714.

In one embodiment, the pigments of the composition are aligned during or after application of the composition. An example of aligning the pigments of the composition is by applying the composition with a magnetic applicator. The magnetic applicator may be used to align the magnetic particles in the cosmetic composition allowing control of their appearance.

General cosmetic compositions may contain preservatives, stabilizers, neutralizing agents, aqueous-phase thickeners (polysaccharide biopolymers, synthetic polymers) or fatty-phase thickeners, such as clay minerals, fillers, perfumes, hydrophilic or lipophilic active substances, surfactants, antioxidants, film-forming polymers, additional colorants, and mixtures thereof. The amounts of these various ingredients are those conventionally employed in the fields in question and, for example, may be from 0.01 to 30% of the total weight of the composition. In one embodiment, the cosmetic composition may further comprise a binder wherein the pigment represents about 0.5% to about 99.5% of the composition.

Lip cosmetic composition may comprise any ingredient usually used in the field concerned, such as water, preferably in an amount ranging from 0 to 95% of the total weight of the composition, water-soluble or liposoluble dyes, antioxidants, essential oils, preserving agents, fragrances, neutralizing agents, liposoluble polymers, in particular hydrocarbon-based polymers such as polyalkylenes or polyvinyl laurate, gelling agents for an aqueous phase, gelling agents for a liquid fatty phase, waxes, gums, surfactants, additional cosmetic or dermatological active agents such as, for example, emollients, moisturizers (for example glycerol), vitamins, liquid lanolin, essential fatty acids, lipophilic or hydrophilic sunscreens, and mixtures thereof. The composition may also contain lipid vesicles of ionic and/or nonionic type. These ingredients (other than the water) may be present in the composition in a proportion of from 0 to 20% of the total weight of the composition.

In one embodiment a coating, ink, or plastic composition contains the pearlescent pigment. In another embodiment an article comprises the pearlescent pigment. A coating, ink, plastic, or article may further comprise a binder, wherein the pigment represents about 0.5% to about 99.5% of the composition, about 0.1% to about 70%, or about 0.2% to about 10%.

In one embodiment an article contains the pearlescent pigment. The article may be printing ink, surface coating, coatings for laser marking, pigment preparation, dry preparation, food colorant, textile coating, architectural coating, synthetic fiber, or fiber based product. The article may be a banknote, cosmetic, automobile, or other object. A coating may be applied to an object as a liquid, vapor, or solid. Examples of methods for applying a coating are by printing, painting, polymeric coating, or spraying. The coating may be a powder, enamel, aerosol, paint, epoxy, or polymer.

The ink may be a magnetic toner. An example of a magnetic toner is one that is used for Magnetic Ink Character Recognition (MICR). These toners may be used to print security codes on checks and are read by low-cost readers. Many of the toners used for MICR are black. The color and magnetic susceptibility of the MICR ink may be adjusted by using different pearlescent pigments.

The arts of making coatings and inks, as well the various printing processes, such as intaglio, flexo, screen, offset, inkjet, gravure, spray-coating, are very well known in the literatures, so it is not repeated here [see “The Printing Ink Manual”, 5^{th} edition, R. H. Leach, ed. Taylor & Francis, Inc.]. Other less common printing processes include digital offset solutions such as the Hewlett-Packard Indigo presses.

Besides the topical applications such as printings or coatings, the pigments can be incorporated directly into substrates during the formation stage to make an article. For example, to a paper, the pigments can be introduced along with other regular paper fillers such as calcite, talc during paper making to fill the open pores of paper near the surface. If the article is a plastic, the pigment can be introduced during the extrusion of substrate. Examples of articles are plastic, glass, ceramic material, concrete, pressed wood, pills, paper, toothpaste, food products, or agricultural products.

The terms goniochromatic, iridescent, and pearlescent, may be used interchangeably to mean a change of color depending on the viewing angle.

Unless otherwise specified, the HPI, CIELAB coordinates, hue angle, h_{ab}, L*, a*, b*, and chroma of a pigment are measured using the pigment drawdown described in Example 1 with a white or black background using a D65 illuminant and a 10 degree observer.

A homogeneous coating of a pigment is a coating that only contains one colored element, it is not a blend of colored elements. An example of a pigment that will not form a homogeneous coating is Cloisonné® Nu Antique Gold pigment, the pigment contains both color pigment and iron oxide.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

### EXAMPLES

### Example 1

### Lustrous, Semi-Opaque Silver Pearlescent Pigment

A suspension of TiO₂ coated natural Mica (60 g, SunPearl Silver White, 10-60 μm particle size) and deionized water (1500 mL) were charged into a 2L jacketed reactor. The mixture was heated to 90° C., and a solution of KNO₃ (1.53 g, in 19.95 mL of deionized water), and a solution of FeSO₄ (5.98 g, in 19.95 mL of deionized water) and H₂SO₄ (1.05 g, conc.) were added to the suspension. A peristaltic pump was used to add NaOH (7.5 g in 42.50 g of deionized water) dropwise until the solution reached a pH 10. The mixture was held at 80° C. for one hour. If necessary, additional NaOH solution was added to hold the pH at 10. The pigment is filtered, rinsed with water, and dried at 60-80° C. A lustrous pigment having a pine green interference color was obtained.

A drawdown of the pigment is prepared to measure the color. The pigment (0.50 g) mixed with acrylic enamel (4.5 g, Delstar DMR 499) at 3000 rpm for 3 min with a high speed mixer (DAC150FVZ-K, Hauschild Engineering). The dispersion is applied to an opacity card (Leneta Form 3B) using a 3 mil (≈76 μm) Bird Applicator. Each drawdown was allowed to dry at room temperature for 30 minutes and then placed in an oven at 60° C. for an additional 30 minutes.

The calorimetric parameters (CIE L*a*b*) of the dried films were measured using a 10 degree observer and D65 illuminant with a 30 mm aperture (specular component included) against both a white and black reference background. The results are shown in Table 2.

### Examples 2 to 9

### Lustrous, Semi-Opaque Silver Pearlescent Pigment

Examples 2 through 9 were made by the procedure of Example 1, except the amounts of the starting materials, the temperature of the reaction, and the final pH are as shown in Table 1. Drawdowns of the pigments were performed as described in Example 1.

TABLE 1
Reaction conditions for Examples 2 through 9.

| | Example | | | | | | | |
|---|---|---|---|---|---|---|---|---|
| | 2 | 3 | 4 | 5 | 6 | 7 | 8 | 9 |
| pH | 10 | 8 | 10 | 8 | 8 | 8 | 10 | 10 |
| Temp | 90 | 80 | 80 | 90 | 90 | 80 | 90 | 80 |
| FeSO₄ | 5.98 | 11.95 | 11.95 | 5.98 | 11.95 | 5.98 | 11.95 | 5.98 |
| DI H₂O | 19.95 | 39.9 | 39.9 | 19.95 | 39.9 | 19.95 | 39.90 | 19.95 |
| KNO₃ | 1.53 | 5.70 | 3.07 | 2.85 | 3.07 | 1.53 | 5.70 | 1.53 |
| DI H₂O | 19.95 | 39.9 | 39.9 | 19.95 | 39.9 | 19.95 | 39.9 | 19.95 |
| H₂SO₄ | 1.09 | 2.11 | 1.26 | 0.63 | 1.26 | 1.05 | 2.11 | 1.05 |
| NaOH | 24.53 | 32.55 | 30.50 | 14.69 | 28.43 | 18.38 | 42.68 | 18.38 |
| added | | | | | | | | |

The temperature and the final pH are two factors that tune b*. The color value b* will be lower when there is a lower final pH, or when the reaction is performed at a higher temperature.

TABLE 2
CIELab values of examples 1 to 9

| | Black Background | | | | | White Background | | | | |
|---|---|---|---|---|---|---|---|---|---|---|
| | | | | | Hue | | | | | Hue |
| | L* | a* | b* | Chroma | angle | L* | a* | b* | Chroma | angle |
| Example 1 | 72.36 | −0.61 | 3.39 | 3.45 | 100.23 | 75.54 | 1.83 | 7.67 | 7.89 | 76.55 |
| Example 2 | 73.74 | −0.91 | 3.25 | 3.37 | 105.69 | 77.28 | 1.52 | 7.77 | 7.92 | 78.94 |
| Example 3 | 65.60 | −0.52 | −0.32 | 0.61 | 211.32 | 66.51 | 0.16 | 0.64 | 0.66 | 75.98 |
| Example 4 | 64.96 | 0 | 1.8 | 1.8 | 89.85 | 65.71 | 0.81 | 2.76 | 2.88 | 73.59 |
| Example 5 | 73.26 | −0.8 | 1.97 | 2.12 | 112.03 | 75.91 | 1.11 | 5.30 | 5.42 | 78.23 |
| Example 6 | 67.39 | −0.82 | −0.68 | 1.06 | 219.46 | 68.08 | −0.16 | 0.40 | 0.43 | 112.20 |
| Example 7 | 74.51 | −0.92 | 2.6 | 2.76 | 109.39 | 78.16 | 1.42 | 6.91 | 7.06 | 78.40 |
| Example 8 | 63.31 | −0.07 | 0.43 | 0.44 | 99.05 | 63.42 | 0.54 | 1.21 | 1.33 | 66.09 |
| Example 9 | 74.68 | −0.69 | 4.13 | 4.19 | 99.49 | 77.98 | 1.94 | 8.75 | 8.96 | 77.56 |

### Example 10

### Neon Blue Pearlescent Pigment

A solution of HCl (706.2 g, 0.1 M solution), FeCl₃ (14.2 g of 45 wt % solution), urea (96 g), and TiO₂ coated natural mica pigment (40 g, SunPearl Iridescent Green, 10-60 μm particle size) is charged to a 1L jacketed pot reactor with agitation at 175 rpm. This initial solution has an approximate pH of 1.1. The solution is then heated to 90° C. to promote the decomposition of urea and a subsequent rise in pH. After about 2 hours at 90° C., the solution pH rises to approximately 6.3-6.5 indicating completion of the batch. For work up, the pigment is filtered off, rinsed with deionized water and dried at 60-80° C.

A neon-like lustrous pigment having a blue interference color combined with a golden yellow absorbance color is obtained.

### Example 11

### Lustrous, Semi-Opaque Blue Pearlescent Pigment

A suspension of the substrate produced in Example 10 (20 g) and deionized water (500 g) were charged into a 1L jacketed reactor. The mixture was heated to 80° C., and a solution of KNO₃ (2.74 g, in 36.5 g of deionized water) and a solution of FeSO₄,7H₂O (10.95 g, in 63.5 g of deionized water) and H₂SO₄ (1.68 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a blue interference color was obtained. The calorimetric measurement of the pigment is measured as in Example 1, and shown in Table 3.

TABLE 3
Color measurement of Example 11

| | Black Background | | | | | White Background | | | | |
|---|---|---|---|---|---|---|---|---|---|---|
| | | | | | Hue | | | | | Hue |
| | L* | a* | b* | Chroma | angle | L* | a* | b* | Chroma | angle |
| Example 11 | 39.86 | −8.59 | −7.51 | 11.41 | 221.15 | 40.33 | −7.82 | −7.02 | 10.51 | 221.90 |

### Example 12

### Lustrous, Semi-Opaque Dark Silver Pearlescent Pigment

A suspension of TiO₂ coated natural Mica (385 g, SunPearl Sparkle White, 10-100 μm particle size) and deionized water (9625 mL) were charged into a 15L jacketed reactor. The mixture was heated to 80° C., and a solution of KNO₃ (77.3 g, in 703 mL of deionized water), and a solution of FeSO₄ (210.8 g, in 703 mL of deionized water) and H₂SO₄ (32.34 g, conc.) were added to the suspension. A peristaltic pump was used to add NaOH (90 g in 510 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C.

A lustrous pigment having a dark silver interference color was obtained. The color of the pigment was measured as in Example 1, see Table 5.

The pigment obtained has a non-conventional ratio Fe(III):Fe(II) of 4:1, as shown in Table 4.

TABLE 4
Titration analysis of Example 12

| Sample | Fe(II) (% by weight) | Fe(III) (% by weight) | Fe(III)/Fe(II) |
|---|---|---|---|
| Example 12 | 1.96 | 7.99 | 4.08 |

TABLE 5
Color measurements of Examples 12 through 22

| | Black Background | | | | | White Background | | | | |
|---|---|---|---|---|---|---|---|---|---|---|
| | L* | a* | b* | C* | h | L* | a* | b* | C* | h |
| Example 12 | 45.97 | 1.45 | 2.69 | 3.05 | 61.73 | 46.14 | 1.67 | 2.94 | 3.38 | 60.36 |
| Example 13 | 45.15 | 1.84 | 3.51 | 3.97 | 62.29 | 45.18 | 2.13 | 3.79 | 4.35 | 60.72 |
| Example 19 | 51.79 | −12.46 | 1.88 | 12.60 | 171.40 | 52.73 | −10.21 | 3.13 | 10.68 | 162.98 |
| Example 20 | 65.82 | −0.53 | 0.81 | 0.97 | 122.97 | 66.34 | 0.43 | 2.23 | 2.27 | 79.20 |
| Example 22 | 50.10 | 0.62 | 0.45 | 0.77 | 36.01 | 50.29 | 0.79 | 0.63 | 1.01 | 38.77 |

### Example 13

### Lustrous, Semi-Opaque Dark Silver Pearlescent Pigment

The procedure followed is analogous to Example 12, except 531.25 g of the NaOH solution was added, and the pH is held at 8. A lustrous pigment having a dark silver interference color was obtained.

The color of the pigment was measured as in Example 1, see Table 5.

As shown in Table 6, example 12 has a higher b* value than example 13. This result confirms the analysis of the calorimetric measurement in the examples 1-9.

TABLE 6
CIELab values of examples 12 and 13

| | Black background | | | | |
|---|---|---|---|---|---|
| Sample | L* | a* | b* | Chroma | Hue angle |
| Example 12 | 44.78 | 1.62 | 2.94 | 3.36 | 61.19 |
| Example 13 | 45.64 | 1.17 | 2.07 | 2.38 | 60.54 |

### Example 14

### Magnetic Alignment of Commercial Pearlescent Pigment

The calorimetric parameters of magnetically aligned and non-aligned Colorona® Blackstar Red, Blue, Green and Gold pigments from Merck were measured as described in example 1. To align the pigments, the opacity card was placed on glass with two circular magnets underneath. The results are shown in Table 7 and Table 8.

### Example 15

### Lustrous, Semi-Opaque Blue Pearlescent Pigment

A suspension of TiO₂ coated natural Mica (20 g, SunPearl Iridescent Blue, 10-60 μm particle size) and deionized water (500 mL) were charged into a 1L jacketed reactor. The mixture was heated to 80° C., and a solution of KNO₃ (2.74 g, in 36.5 mL of deionized water) and a solution of FeSO₄ (10.95 g, in 36.5 mL of deionized water) and H₂SO₄ (1.68 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a blue interference color was obtained. The color of the pigment was measured as in Example 14, see Table 7 and Table 8.

### Example 16

### Lustrous, Semi-Opaque Violet Pearlescent Pigment

The procedure followed is analogous to Example 15, except 20 g of TiO₂ coated natural Mica (SunPearl Iridescent Red, 10-60 μm particle size) was used. A lustrous pigment having a violet interference color was obtained.

A drawdown of the pigment obtained was prepared as describe in the example 14, see Table 7 and Table 8.

### Example 17

### Lustrous, Semi-Opaque Gold Pearlescent Pigment

A suspension of TiO₂ coated natural Mica (25 g, SunPearl Iridescent Gold, 10-60 μm particle size) and deionized water (625 mL) were charged into a 1L jacketed reactor. The mixture was heated to 80° C., and a solution of KNO₃ (5.02 g, in 45.63 mL of deionized water), and a solution of FeSO₄ (13.68 g, in 45.63 mL of deionized water) and H₂SO₄ (2.10 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a gold interference color was obtained. The color of the pigment was measured as in Example 14, see Table 7 and Table 8.

### Example 18

### Lustrous, Semi-Opaque Green Pearlescent Pigment

The procedure followed is analogous to Example 17, except 25 g of TiO₂ coated natural Mica (SunPearl Iridescent Green, 10-60 μm particle size) was used. A lustrous pigment having a green interference color was obtained.

A drawdown of the pigment obtained was prepared as describe in the example 14, see Table 7 and Table 8.

TABLE 7
Aligned and un-aligned CIELab values of examples 15 to 18 with a black background

| | | Un-aligned pigments | | | | | Aligned pigments | | | | | |
|---|---|---|---|---|---|---|---|---|---|---|---|---|
| | | | | | | Hue | | | | | Hue | |
| | | L* | a* | b* | C | angle | L* | a* | b* | C | angle | ΔE* |
| Example | Blue | 37.37 | −4.77 | −11.86 | 12.79 | 248.09 | 27.89 | 0.49 | −0.66 | 0.82 | 306.51 | 15.59 |
| 15 | | | | | | | | | | | | |
| Colorona ® | Blue | 29.84 | −1.01 | −3.88 | 4.01 | 255.41 | 29.65 | −1.03 | −3.67 | 3.81 | 254.25 | 0.29 |
| Blackstar | | | | | | | | | | | | |
| Blue | | | | | | | | | | | | |
| Example | Violet | 34.73 | 11.64 | −5.79 | 13 | 333.53 | 28.21 | 1.94 | 0.15 | 1.94 | 4.32 | 13.11 |
| 16 | | | | | | | | | | | | |
| Colorona ® | Violet | 32.73 | 7.74 | 1.73 | 7.93 | 12.61 | 29.99 | 5.16 | 0.98 | 5.25 | 10.78 | 3.84 |
| Blackstar | | | | | | | | | | | | |
| Red | | | | | | | | | | | | |
| Example | Gold | 42.83 | 3.46 | 16.03 | 16.40 | 77.80 | 30.03 | 0.86 | 2.69 | 2.82 | 72.33 | 18.67 |
| 17 | | | | | | | | | | | | |
| Colorona ® | Gold | 42.52 | 8.92 | 16.56 | 18.81 | 61.68 | 38.29 | 7.24 | 12.78 | 14.69 | 60.48 | 5.92 |
| Blackstar | | | | | | | | | | | | |
| Gold | | | | | | | | | | | | |
| Example | Green | 42.18 | −7.14 | 6.52 | 9.67 | 137.59 | 28.77 | −0.28 | 0.72 | 0.77 | 110.91 | 16.14 |
| 18 | | | | | | | | | | | | |
| Colorona ® | Green | 33.40 | −4.08 | −2.20 | 4.63 | 208.40 | 27.21 | −0.84 | −1.59 | 1.80 | 208.40 | 7.01 |
| Blackstar | | | | | | | | | | | | |
| Green | | | | | | | | | | | | |

TABLE 8
Aligned and un-aligned CIELab values of examples 15 to 18 with a white background

| | | Un-aligned pigments | | | | | Aligned pigments | | | | | |
|---|---|---|---|---|---|---|---|---|---|---|---|---|
| | | | | | | Hue | | | | | Hue | |
| | | L* | a* | b* | C | angle | L* | a* | b* | C | angle | ΔE* |
| Example 15 | Blue | 37.72 | −3.94 | −11.24 | 11.91 | 250.65 | 29.42 | 0.91 | −0.88 | 1.27 | 315.82 | 14.13 |
| Colorona ® | Blue | 30.57 | −1.13 | −4.14 | 4.29 | 254.78 | 29.00 | −0.71 | −3.34 | 3.41 | 257.98 | 1.81 |
| Blackstar Blue | | | | | | | | | | | | |
| Example 16 | Violet | 35.12 | 11.55 | −5.28 | 12.70 | 335.41 | 28.98 | 2.17 | 0.98 | 2.38 | 24.27 | 12.84 |
| Colorona ® | Violet | 32.55 | 7.65 | 1.78 | 7.86 | 13.10 | 29.83 | 5.05 | 1.02 | 5.15 | 11.44 | 3.85 |
| Blackstar Red | | | | | | | | | | | | |
| Example 17 | Gold | 42.87 | 3.45 | 16.06 | 16.42 | 77.88 | 29.72 | 1.06 | 2.44 | 2.66 | 66.56 | 19.08 |
| Colorona ® | Gold | 41.57 | 8.61 | 15.80 | 17.99 | 61.42 | 46.34 | 10.02 | 19.66 | 22.07 | 62.99 | 6.30 |
| Blackstar Gold | | | | | | | | | | | | |
| Example 18 | Green | 42.38 | −6.26 | 6.84 | 9.27 | 132.45 | 29.57 | 1.51 | 2.36 | 2.80 | 57.34 | 15.63 |
| Colorona ® | Green | 32.59 | −3.56 | −2.05 | 4.11 | 209.96 | 26.19 | −0.28 | −1.24 | 1.27 | 257.18 | 7.24 |
| Blackstar Green | | | | | | | | | | | | |

As shown in the calorimetric data of Table 7 and Table 8, examples 15 to 18 show a brighter and lighter color than the commercially available pigments. Examples 15 to 18 also exhibit also a higher ΔE* between the un-aligned and aligned pigments compared to the commercially available pigments.

### Example 19

### Lustrous, Semi-Opaque Green Pearlescent Pigment

A suspension of TiO₂ coated natural Mica (30 g, SunPearl Iridescent Green, 10-60 μm particle size) and deionized water (750 mL) were charged into a 1L jacketed reactor. The mixture was heated to 80° C., and a solution of KNO₃ (3.03 g, in 27.35 mL of deionized water), and a solution of FeSO₄ (8.25 g, in 27.35 mL of deionized water) and H₂SO₄ (1.25 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a green interference color was obtained. The color of the pigment was measured as in Example 1, see Table 10. The magnetic susceptibility was measured, see Table 11. The Fe(III):Fe(II) ratio was measured, see Table 9—Fe(III):Fe(II) ratios of Examples 19 and 19A.

### Example 19A

### Comparison Example

A suspension of TiO₂ coated natural Mica (30 g, SunPearl Iridescent Green, 10-60 μm particle size) and deionized water (750 mL) were charged into a 1L jacketed reactor. The mixture was heated to 80° C. A solution of KNO₃ (3.03 g, in 27.35 mL of deionized water), and a solution of FeSO₄ (8.25 g, in 27.35 mL of deionized water) and H₂SO₄ (1.25 g, conc.) were added simultaneously to the suspension. During the addition a pH of 8 was kept constant by the addition of a NaOH solution (7.5 g in 42.50 g of deionized water). After one hour, from the start of addition of the solutions, the pigment is filtered, rinsed with water, and dried at 60-80° C. A lustrous pigment having a green interference color was obtained. The color of the pigment was measured as in Example 1, see Table 10. The Fe(III):Fe(II) ratio was measured, see Table 9—Fe(III):Fe(II) ratios of Examples 19 and 19A.

TABLE 9
Fe(III):Fe(II) ratios of Examples 19 and 19A

| | Fe | | | |
|---|---|---|---|---|
| | (% by | Fe(II) | Fe(III) | |
| | weight) | (% by weight) | (% by weight) | Fe(III):Fe(II) |
| Example 19 | 5.63 | 1.36 | 4.27 | 3.14 |
| Example 19A | 4.9 | 2.05 | 2.85 | 1.39 |

TABLE 10
CIELab values of examples 19 and 19A

| | Black background | | | | | White background | | | | |
|---|---|---|---|---|---|---|---|---|---|---|
| | L* | a* | b* | C* | h | L* | a* | b* | C* | h |
| Example 19 | 51.79 | −12.46 | 1.88 | 12.60 | 171.40 | 52.73 | −10.21 | 3.13 | 10.68 | 162.98 |
| Example 19A | 49.60 | −11.32 | 3.18 | 11.76 | 164.32 | 50.50 | −9.24 | 4.40 | 10.21 | 154.44 |

### Example 20

### Lustrous, Opaque Silver Pearlescent Pigment

A suspension of TiO₂ coated natural Mica (60 g, SunPearl Silver White, 10-60 μm particle size) and deionized water (1500 mL) were charged into a 2L jacketed reactor. The mixture was heated to 80° C., and a solution of KNO₃ (3.84 g, in 34.83 mL of deionized water), and a solution of FeSO₄ (10.45 g, in 34.83 mL of deionized water) and H₂SO₄ (1.59 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a silver interference color was obtained. The magnetic susceptibility was measured, see Table 11.

### Example 21

### Lustrous, Semi-Opaque Yellow Pearlescent Pigment

A suspension of coated natural Mica (30 g, CQV, Soliens Soft Gold, K particles) and deionized water (750 mL) were charged into a 1L jacketed reactor. The mixture was heated to 80° C., and a solution of KNO₃ (0.41 g, in 5.47 mL of deionized water), and a solution of FeSO₄ (1.65 g, in 5.47 mL of deionized water) and H₂SO₄ (0.25 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a yellow gold interference color was obtained. The magnetic susceptibility was measured, see Table 11.

### Example 22

### Lustrous, Semi-Opaque Silver Pearlescent Pigment

A suspension of coated natural Mica (25 g, SunPearl Silver White, 10-60 μm particle size) and deionized water (625 mL) were charged into a 1L jacketed reactor. The mixture was heated to 80° C., and a solution of KNO₃ (5.02 g, in 45.63 mL of deionized water), and a solution of FeSO₄ (13.69 g, in 45.63 mL of deionized water) and H₂SO₄ (2.10 g, conc.) were added to the suspension. A peristaltic pump was used to add NaOH (7.5 g in 42.50 g of deionized water) dropwise until the solution reached a pH 8. The mixture was held at 80° C. for one hour. If necessary, additional NaOH solution was added to hold the pH at 8. The pigment is filtered, rinsed with water, and dried at 60-80° C. A lustrous pigment having a silver interference color was obtained. The magnetic susceptibility was measured, see Table 11.

The magnetic susceptibility of the pigments increases with the ratio of Fe/substrate (by weight).

TABLE 11
Magnetic susceptibility of Examples 19-22

| | | | Mass |
|---|---|---|---|
| | | Fe/Substrate | Susceptibility |
| Sample | Color | By weight | SI × 10⁻⁵ m³/kg |
| Example 19 | Pine green | 0.055 | 3.150 |
| Example 20 | Silver | 0.035 | 1.880 |
| Example 22 | silver | 0.110 | 7.267 |

### Example 23

### Clear Gel Lip Gloss Preparation

The constituents of the clear gel lip gloss base shown in Table 12 are mixed homogeneously and heated to 80° C. Following sufficient cooling to room temperature, the pigment from Example 20 was added at 2 wt % to the base gel and mixed thoroughly.

TABLE 12
Composition of clear gel lip gloss base

| Ingredients | Weight Fraction (%) |
|---|---|
| Versagel ME750 (Penreco) | 81.5 |
| Ceraphyl 368 (Sblack, ISP) | 10 |
| Ceraphyl 55 (ISP) | 5 |
| Isostearyl Isostearate (Mosselman) | 3 |
| Germaben (Clariant) | 0.5 |

### Example 24

### Eye Shadow Cream

Xanthan gum and magnesium aluminum silicate were dispersed into deionized water using high shear mixing until the mixture was smooth, to form Phase A. Triethanolamine, propylene glycol, and a water soluble preservative (Phase B) were added to the smooth gum mixture of Phase A and mixed until smooth. Stearic acid, glyceril stearate, and oleyl alcohol were heated to 75±5° C. with gentle agitation, to form Phase D.

The pearlescent pigment material (Phase C) was added to the Phase A-B mixture with gentle agitation, and maintained at a temperature of 75±5° C. Phase D was added to the Phase A-B-C mixture with gentle agitation, while maintaining a temperature of 75±5° C. A constant agitation was maintained and the overall mixture was cooled to 35±5° C.

The resulting eye shadow cream is a shimmering dark silver shade with gold, red and green iridescent sparkling points, depending on the viewing angle.

TABLE 13
Composition of the eye shadow cream

| Ingredients | Weight Fraction (%) |
|---|---|
| Phase A | |
| Water | (q.s. to 100%) 65.10 |
| Magnesium Aluminum Silicate | 1.00 |
| Xanthan Gum | 0.30 |
| Phase B | |
| Triethanolamine (TEA 99%) | 0.30 |
| Propylene Glycol | 8.00 |
| Preservative (Water soluble) | q.s. |
| Phase C | |
| Example 12 | 20.00 |
| Phase D | |
| Stearic Acid (Stearic Acid 94%) | 4.00 |
| Glyceril Stearate | 0.80 |
| Oleyl Alcohol | 0.50 |

### Example 25

### Pressed Powder

Talc, dimethicone/dimethicone crosspolymer, and preservatives were thoroughly blended and dispersed using appropriate dry blending/dispersing equipment. The pearlescent pigment material of Phase B was added to the dry blended ingredients and mixed until uniform.

The resulting press powder is characterized by a lustrous, green-shade pearlescent appearance with gold, red and green iridescent sparkling points depending on the viewing angle.

TABLE 14
Composition of the pressed powder

| Ingredients | Weight Fraction (%) |
|---|---|
| Phase A | |
| Talc | 45-80 (q.s to 100) |
| Dimethicone and Dimethicone | 5.00 |
| Crosspolymer | |
| Preservatives | (q.s to 100) |
| Phase B | |
| Example 19 | 15.00-50.00 |

### Example 26

### Nail Polish

Pearlescent pigment from example 20 (5 parts) was mixed with the nail polish base (95 parts, see Table 9) in an appropriate size vessel fitted with a Lightning™ type propeller mixer. The components were mixed until uniform.

The resulting nail enamel and nail laquer is characterized by a high-chroma lustrous, light silver pearlescent appearance with gold, red and green iridescent sparkling points depending on the viewing angle.

TABLE 15
Nail polish base

| Ingredients | Weight Fraction (%) |
|---|---|
| Butyl Acetate | 25-50 |
| Ethyl Acetate | 10-25 |
| Nitrocellulose | 10-25 |
| Acetyl Tributyl Citrate | 5-10 |
| Phtalic Anhydride/Trimellitic | 5-10 |
| Anhydride/Glycol Copolymer | |
| Isopropyl Alcohol | 5-10 |
| Stearalkonium Hectorite | 1-5 |
| Adipic Acid/Fumaric Acid/Phtalic | 1-5 |
| Acid/Tricyclodecane Dimethanol Copolymer | |
| Citric Acid | <0.1 |

### Example 27

### High Gloss, Colored Lipstick

Castor oil, isononyl isononanoate, pentaerythrityl tetracaprylate/tetracaprate, octyldodecanol, lanolin oil, caprylic/capric/stearic triglyceride, candelilla wax, carnauba wax, polybutene H-100, ozokerite, lanolin wax, red 7 lake, preservative, and antioxidant were all weighed and placed into a heated vessel. The temperature was raised to 85±3° C. The ingredients were stirred until they were melted and uniform.

The pearlescent pigment of Phase B was dispersed in the castor oil of Phase A then milled in either a colloid or roller mill. The dispersed pigment was then added and mixed with the remainder of Phase A. The fragrance of Phase C was then added and mixed with constant stirring. The composition was poured at 75±5° C. then molded, cooled, and flamed into lipstick.

The resulting lipstick is characterized by a high-chroma lustrous, green pearlescent appearance.

TABLE 16
Composition of the high gloss, colored lipstick

| Ingredients | Weight Fraction (%) |
|---|---|
| Phase A | |
| Castor Oil | (q.s to 100%) 14.56 |
| Isononyl Isononanoate | 17.51 |
| Pentaerythrityl TetraCaprylate/Tetracaprate | 8.75 |
| Octyldodecanol | 5.47 |
| Lanolin Oil | 11.93 |
| Caprylic/Capric/Stearic Triglyceride | 7.11 |
| Candelilla Wax | 9.30 |
| Carnauba Wax | 3.28 |
| Polybutene H-100 | 7.66 |
| Ozokerite | 2.20 |
| Lanolin Wax | 1.09 |
| Red 7 Lake | 0.80 |
| Preservative | q.s. |
| Antioxidant | q.s. |
| Phase B | |
| Example 19 | 10.00 |
| Phase C | |
| Fragrance | 0.10 |

### Example 28

### High Gloss Lipstick

Castor oil, isononyl isononanoate, pentaerythrityl tetracaprylate/tetracaprate, octyldodecanol, lanolin oil, caprylic/capric/stearic triglyceride, candelilla wax, carnauba wax, polybutene H-100, ozokerite, lanolin wax, preservative, and antioxidant were all weighed and placed into a heated vessel. The temperature was raised to 85±3° C. The ingredients were stirred until they were melted and uniform.

The pearlescent pigment of Phase B was dispersed in the castor oil of Phase A then milled in either a colloid or roller mill. The dispersed pigment was then added and mixed with the remainder of Phase A. Fragrance from Phase C was then added and mixed with constant stirring. The composition was poured at 75±5° C., then molded, cooled and flamed into lipstick.

The resulting lipstick is characterized by a lustrous light silver pearlescent appearance.

TABLE 17
Composition of the high gloss, colored lipstick

| Ingredients | Weight Fraction (%) |
|---|---|
| Phase A | |
| Castor Oil | (q.s to 100%) 15.36 |
| Isononyl Isononanoate | 17.51 |
| Pentaerythrityl TetraCaprylate/Tetracaprate | 8.75 |
| Octyldodecanol | 5.47 |
| Lanolin Oil | 11.93 |
| Caprylic/Capric/Stearic Triglyceride | 7.11 |
| Candelilla Wax | 9.30 |
| Carnauba Wax | 3.28 |
| Polybutene H-100 | 7.66 |
| Ozokerite | 2.20 |
| Lanolin Wax | 1.09 |
| Preservative | q.s. |
| Antioxidant | q.s. |
| Phase B | |
| Example 20 | 10.00 |
| Phase C | |
| Fragrance | 0.10 |

### Example 29

### Clear Gel Lip Gloss

Hydrogenated polyisobutene, ethylene/propylene/styrene copolymer, butylene/ethylene/styrene copolymer, ethylhexyl palmitate, tridecyl neopentanoate, isostearyl isostearate, and preservative were all weighed and placed into a heated vessel. The temperature was raised to 50±3° C. The ingredients were stirred until they were melted and uniform. At room temperature, pearlescent pigment of Phase B was added to Phase A and mixed until all the pearlescent pigment was well dispersed. Fragrance may be added if needed, and mixed with constant stirring. The composition was poured at room temperature.

The resulting lip gloss is characterized by a dark silver pearlescent appearance with gold, red and green iridescent sparkling points depending on the viewing angle.

TABLE 18
Composition of the clear gel lip gloss

| Ingredients | Weight Fraction (%) |
|---|---|
| Phase A | |
| Hydrogenated Polyisobutene and | 73.35 |
| Ethylene/Propylene/Styrene Copolymer and | |
| Butylene/Ethylene/Styrene Copolymer | |
| Ethylhexyl Palmitate | 9.00 |
| Tridecyl Neopentanoate | 4.50 |
| Isostearyl Isostearate | 2.70 |
| Preservative | q.s. |
| Phase B | |
| Example 12 | 10.00 |

### Prophetic Example 30

### Nail Varnish

The cosmetic composition of a nail lacquer comprising a pearlescent pigment may be prepared from the components set forth in Table 19.

TABLE 19
Nail varnish

| Ingredients | Weight Fraction (%) |
|---|---|
| Nail polish base (Kirker Enterprises, Inc. of | 94 |
| Patterson, NJ) | |
| Example 16 | 6 |

### Prophetic Example 31

### Mascara

The cosmetic composition of a mascara comprising a pearlescent pigment may be prepared from the components set forth in Table 20.

TABLE 20
Mascara

| Ingredients | Amount (g) |
|---|---|
| Petroleum Distillate | 68 |
| Polyethylene | 12 |
| Dihydroabietyl alcohol | 5 |
| Candelilla wax | 2.4 |
| Aluminum stearate | 0.05 |
| Butylparaben | 0.1 |
| Black iron oxide | 4 |
| Example 15 | 8 |

### Prophetic Example 32

### Face Powder

The cosmetic composition of a face powder comprising a pearlescent pigment may be prepared from the components set forth in Table 21.

TABLE 21
Face Powder

| Ingredients | Amount (g) |
|---|---|
| Iron oxide | 6.57 |
| Zinc stearate | 4 |
| Titanium dioxide | 2 |
| Bismuth oxychloride | 10 |
| Nylon powder sold under the name | 20 |
| “ORGASOL ®” by the company | |
| ATOCHEM | |
| Vaseline oil | 3.26 |
| Oleyl alcohol | 0.6 |
| Isopropyl myristate | 0.43 |
| Propyl para-hydroxybenzoate | 0.12 |
| Example 21 | 20 |
| Talc qs. | 80 |

### Prophetic Example 33

### Eye Shadow

The cosmetic composition of an eye shadow comprising a pearlescent pigment may be prepared from the components set forth in Table 22.

TABLE 22
Eye shadow

| Ingredients | Weight Fraction (%) |
|---|---|
| Talc | 49.75 |
| Titanium dioxide | 1 |
| Zinc stearate | 5 |
| Red iron oxide | 0.15 |
| Yellow iron oxide | 0.1 |
| Polyethylene | 3 |
| Magnanese violet | 5 |
| Example 22 | 25 |
| Mineral oil | 7 |
| Dimethicone fluid | 4 |

### Prophetic Example 34

### Blush

The cosmetic composition of a blush comprising a pearlescent pigment may be prepared from the components set forth in Table 23.

TABLE 23
Blush

| Ingredients | Amount (g) |
|---|---|
| Zinc stearate | 3 |
| Titanium oxide | 2 |
| Iron oxide | 9 |
| Mica | 24 |
| Nylon powder sold under the name | 15 |
| ORGASO ® by the company ATOCHEM | |
| Example 21 | 5 |
| Vaseline oil | 3.26 |
| Oleyl alcohol | 0.6 |
| Isopropyl myristate | 0.43 |
| Propyl para-hydroxybenzoate | 0.12 |
| Talc qs. | 100 |

### Prophetic Example 35

### Hair and Body Gel

The cosmetic composition of a hair and body gel comprising a pearlescent pigment may be prepared from the components set forth in Table 24.

TABLE 24
Hair and Body Gel

| Ingredients | Weight Fraction (%) |
|---|---|
| deionized water | 84 |
| Carbomer | 2 |
| Example 20 | 7.8 |
| Glycerin | 2.5 |
| Vinylpyrrolidone/vinyl actetate | 2.5 |
| copolymer Triethanolamine | 1 |
| Germaben-11 ® | 0.2 |

### Prophetic Example 36

### Lotion

The cosmetic composition of a lotion comprising a pearlescent pigment may be prepared from the components set forth Table 25.

TABLE 25
Lotion

| Ingredients | Amount (g) |
|---|---|
| deionized water | 79.6 |
| Carbomer | 0.5 |
| Polysorbate | 0.8 |
| Propylene glycol | 2 |
| Glycerin | 5 |
| Triethanolamine | 0.6 |
| Example 17 | 2 |
| Acetylated lanolin alcohol | 3 |
| Cetyl alcohol | 2 |
| Stearic acid | 5 |
| LiquaPar ® | 0.5 |

### Prophetic Example 37

### Foundation

The cosmetic composition of a foundation comprising a pearlescent pigment may be prepared from the components set forth in Table 26.

TABLE 26
Foundation

| Ingredients | Amount (g) |
|---|---|
| Glycerol stearate | 2.2 |
| Triglycerides of capric/caprylic acids sold under the | 15.0 |
| name “MIGLYOL 812 ®” by | |
| the company DYNAMIT NOBEL | |
| Yellow iron oxides | 0.75 |
| Brown iron oxides | 0.47 |
| Black iron oxide | 0.23 |
| Titanium dioxide | 4.55 |
| Methyl para-hydroxybenzoate | 0.1 |
| Propyl para-hydroxybenzoate | 0.1 |
| Imidazolidinyl urea | 0.3 |
| 2-hydroxy-4-methoxybenzophenone | 0.5 |
| Octyl N,N-dimethylparaaminobenzoate. | 0.5 |
| Pearlescent pigment example 21 | 3.0 |
| Aluminum and magnesium silicate sold under the name | 1.0 |
| “VEEGUM ®”by the company VANDERBILT | |
| Triethanolamine | 1.0 |
| Cellulose gum | 0.16 |
| Aluminum salt of the product of the reaction of | 5.0 |
| octenylsuccinic anhydride with starch sold under | |
| the name “DRY FLO ®” by the company | |
| NATIONAL STARCH | |
| Cyclomethicone sold under the name “VOLATIL | 10.0 |
| SILICONE 7158 ®” by the company | |
| UNION CARBIDE | |
| Water | 47.34 |
| Propylene glycol | 2.0 |
| Glycerin | 3.0 |
| Sodium salt of lauroylsarcosine sold under the name | 0.6 |
| “ORAMIX L30 ®”by the company SEPPIC | |
| Stearic acid | 2.2 |

1. A pearlescent pigment made by a process comprising:
providing a substrate,
coating iron oxide on the substrate by oxidizing an iron salt with an oxidant in a mixture while increasing the pH of the mixture from acidic to basic;
wherein the iron oxide coating formed has a Fe(III)/Fe(II) ratio of greater than 2.

2. The pearlescent pigment formed by the process of claim 1, wherein the pearlescent pigment has a magnetic mass susceptibility of greater than about 0.05×10⁻⁵ m³/kg.

3. The pearlescent pigment formed by the process of claim 1, wherein the oxidant is selected from the group consisting of nitrates and chlorates.

4. The pearlescent pigment formed by the process of claim 1, wherein the pH of the mixture is increased from a range of about 0.2 to about 4 to a range of about 7 to about 12.

5. The pearlescent pigment formed by the process of claim 1, wherein the iron salt is selected from the group consisting of ammonium iron(II) sulfate, iron(II) halides, iron(II) sulfate, iron(II) acetates, iron(II) carbonates, and iron(II) phosphates.

6. The pearlescent pigment formed by the process of claim 1, wherein the substrate is selected from the group consisting of natural mica, synthetic mica, glass flakes, Al₂O₃ platelets, SiO₂ platelets, BiOCl, borosilicate, synthetic alumina, and boron nitride.

7. The pearlescent pigment formed by the process of claim 1, wherein the pearlescent pigment further comprises a second layer located between the substrate and the first layer, wherein the second layer comprises an oxide selected from the group consisting of TiO₂, Fe₂O₃, FeOOH, ZrO₂, SnO₂, Cr₂O₃, SiO₂, BiOCl, and ZnO.

8. The pearlescent pigment of claim 1, wherein the mean particle size is from about 1 μm to about 750 μm and the mean thickness of the pigment is from about 0.05 μm to about 5 μm.

9. The pearlescent pigment of claim 1, wherein the pigment further has an outer protective coating.

10. The pearlescent pigment of claim 1, wherein the pigment has a ΔE* of magnetically aligned and non-aligned homogeneous coatings of the pigment, measured over a white background, not less than 10.

11. The pearlescent pigment of claim 1, wherein the iron of one coating of iron oxide is less than about 15% of the weight of the pigment.

12. A process for making a pearlescent pigment comprising:
providing a substrate,
coating iron oxide on the substrate by oxidizing an iron salt with an oxidant in a mixture while increasing the pH from acidic to basic.

13. The process of claim 12, wherein the oxidant is selected from the group consisting of nitrates and chlorates.

14. The process of claim 12, wherein the pH of the mixture is increased from a range of about 0.2 to about 4 to a range of about 7 to about 12.

15. The process of claim 12, wherein the iron salt is selected from the group consisting of ammonium iron(II) sulfate, iron(II) halides, and iron(II) sulfate.

16. The process of claim 12, wherein the pH is increased by addition of a base selected from the group consisting of NaOH, KOH, LiOH, urea, and ammonia.

17. A pearlescent pigment, wherein the pigment is an inorganic material and the color of a homogeneous coating of the pigment, measured over a black background, has:
a CIELAB hue angle, h_{ab}, from about 40 to about 320 degrees, wherein L* is from about 35 to about 80, and the chroma value is less than 5.

18. The pigment of claim 17, wherein the pigment has a ΔE* of magnetically aligned and non-aligned homogeneous coatings of the pigment, measured over a white background, not less than 10.

19. The pigment of claim 17, wherein the pigment has an iron oxide coating wherein the iron of the iron oxide has a Fe(III)/Fe(II) ratio of greater than about 2, and a magnetic mass susceptibility of greater than about 0.05×10⁻⁵ m³/kg.

20. A coating, ink, plastics, or cosmetic composition containing the pigment of claim 1.

21. The composition of claim 20, further comprising a binder, wherein the pigment represents about 0.5% to about 99.5% of the composition by weight.

22. The cosmetic composition of claim 20, wherein cosmetic composition is selected from the group consisting of a nail polish, lipstick, lip gloss, mascara, body powder, face powder, eye shadow, hair gel, body gel, hair wash, body wash, lotion, and foundation.

23. A method for applying the cosmetic composition of claim 20, wherein the pigments of the composition are magnetically aligned during or after application of the composition.

24. An article comprising the pigment of claim 1.

25. The article of claim 24, wherein the article is selected from the group consisting of: printing ink, surface coating, coatings for laser marking, pigment preparation, dry preparation, food colorant, textile coating, architectural coating, synthetic fiber, fiber based product, and MICR toner.
